(19) United States
(12) Patent Application Publication
(10) Pub. No.: US 2024/0325696 A1
(43) Pub. Date: Oct. 3, 2024

(54) AXIAL ACTUATION MECHANISM FOR CATHETERS

(71) Applicant: BIOTRONIK AG, Buelach (CH)

(72) Inventors: Martial Willimann, Schlatt (CH); Sebastian Klaus, Embrach (CH)

(21) Appl. No.: 18/580,515

(22) PCT Filed: Aug. 9, 2022
(86) PCT No.: PCT/EP2022/072289

§ 371 Date: Jan. 18, 2024

(30) Foreign Application Priority Data
Aug. 10, 2021 (EP) 21190660.7
Jan. 25, 2022 (EP) 22153071.0

(51) Int. Cl. A61M 25/10; A61M 25/06; A61M 25/09

(52) U.S. Cl. CPC A61M 25/104; A61M 25/09041; A61M 25/0625; A61M 2025/0681

(57) ABSTRACT

A catheter system includes an outer catheter having an outer catheter shaft defining an outer catheter lumen. At least one inner member is arranged within the lumen. The at least one inner member is relatively movable to the outer catheter in an unrestricted moving state. An axial movement restriction device includes an actuation mechanism and a locking mechanism. The axial movement restriction device can restrict an axial movement of the at least one inner member in a restricted moving state compared to the axial movement in the unrestricted moving state.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/072289, which was filed Aug. 9, 2022, which application claimed priority from EP application Ser. No. 21/190,660.7, which was filed Aug. 10, 2021, and from EP application Ser. No. 22/153,071.0, which was filed Jan. 25, 2022.

### FIELD OF THE INVENTION

A field of the invention concerns The present invention relates to a catheter system for the treatment of vascular (e.g. coronary) or non-vascular conditions or diseases, for example for treating stenoses, occlusions, lesions, for therapeutic purposes as well as for applying drug or fluid media. The catheter system includes an axial actuation mechanism e.g. for mechanical probing or channeling of stenoses as well as applying drug or media. The invention further relates to a method for operating such a catheter system. The invention also relates to an axial movement restriction device for restricting an axial movement of at least one inner member being arranged in a lumen of an outer catheter.

### BACKGROUND

Certain clinical interventional procedures, for example related to diagnosis and therapy of stenoses are often demanding when the patient anatomy or vessel morphology is complex.

### SUMMARY OF THE INVENTION

Preferred catheter systems can provide several functionalities, including improved guidewire negotiations (maneuvering through the vascular system), lesion penetrations and lesion recanalizations as well as application of media or drugs. A preferred catheter system includes or consisting of an outer catheter having an outer catheter shaft defining an outer catheter lumen. At least one inner member is arranged within the lumen. The at least one inner member is relatively movable with respect to the outer catheter in an unrestricted moving state. An axial movement restriction device includes an actuation mechanism and a locking mechanism. The axial movement restriction device is configured to restrict axial movement of the at least one inner member in a restricted moving state and permit axial movement in the unrestricted moving state. The actuation mechanism is one of a rotation-translation gear, a ball screw drive actuation mechanism or a spindle driven actuation mechanism.

### BRIEF DESCRIPTION OF THE DRAWINGS

Features and advantages of the present invention and embodiments thereof shall be explained in the following figures, wherein

FIG. 1 shows an embodiment of a catheter system,

FIG. 2 shows an embodiment of a support catheter,

FIG. 3 shows an embodiment of a dilator,

FIG. 4 shows an embodiment of a balloon catheter,

FIG. 5 shows a schematic drawing of a catheter system including an axial movement restriction device,

FIG. 6 shows another embodiment of a catheter system including an axial movement restriction device in a cross-sectional side view,

FIG. 7 shows an embodiment of a locking mechanism in a cross-sectional side view,

FIG. 8 shows an embodiment of a catheter system with a locking mechanism in a cross-sectional side view,

FIG. 9 shows a further embodiment of a locking mechanism in a side view,

FIG. 10 shows another embodiment of a catheter system with an axial movement restriction device in a cross-sectional side view,

FIG. 11 shows a schematic drawing of an axial movement restriction device including an actuation mechanism and a locking mechanism,

FIG. 12 shows another embodiment of an axial movement restriction device including in a cross-sectional side view,

FIG. 13 shows another embodiment of a catheter system including an axial movement restriction device in a cross-sectional side view,

FIG. 14 shows a part of the axial movement restriction device of FIG. 13 in a cross-sectional view.

FIG. 15 shows a part of the axial movement restriction device of FIG. 13 in a cross-sectional front view,

FIG. 16 shows a part of the axial movement restriction device of FIG. 13 in a cross-sectional front view,

FIG. 17A-17C show another embodiment of an axial movement restriction device,

FIG. 18 shows the axial movement restriction device of FIG. 13 in a cross-sectional side view.

### DETAILED DESCRIPTION OF THE PREFERRED EMBODIMENTS

A preferred catheter system includes an outer catheter and an inner (tubular) member. The outer catheter has an outer catheter shaft defining a (tubular) lumen capable of receiving an inner (tubular) member. The outer catheter has a proximal outer catheter end and a distal outer catheter end. The distal outer catheter end is the end which is inserted into a human or animal body. The proximal outer catheter end is the end, which is operated by an operator, in particular an interventional physician e.g. cardiologist or radiologist.

The outer catheter may be a support catheter.

The inner (tubular) member and the outer catheter (e.g. support catheter) are relatively movable to each other in an unrestricted moving state in axial and (in a very limited manner) in radial direction. Thus, in the unrestricted moving state an axial movement of the inner member is enabled.

The catheter system includes an axial movement restriction device. The axial movement restriction device enables a restriction of the axial movement of the at least one inner member in a restricted moving state compared to the axial movement in the unrestricted moving state.

The axial movement restriction device includes a locking mechanism and an actuation mechanism. The locking mechanism and the actuation mechanism are connected with each other or coupled to each other. Thus, the locking mechanism and the actuation mechanism are arranged together. The axial movement restriction device can include a locking mechanism including the locking mechanism and an actuation mechanism including the actuation mechanism, wherein the locking mechanism and the actuation mechanism are connected with each other or coupled to each other.

The axial movement restriction device also enables an unrestricted axial movement of the inner member in the unrestricted moving state with respect to the outer catheter (e.g. support catheter). It is to be understood by a skilled person that the unrestricted axial movement of the inner member is nevertheless limited by the catheter design.

The locking mechanism if taken individually would enable a locking of the inner member in the restricted moving state with respect to the outer catheter (e.g. support catheter) in a selectable position and the axial actuation mechanism if taken individually would enable an axial movement of the (at least one) inner member and the outer catheter relative to each other in the restricted moving state. Thus, the locking mechanism and the actuation mechanism together (or clearly spoken the movement restriction mechanism) enable a restricted axial movement of the (at least one) inner member and the outer catheter relative to each other in the restricted moving state. Therefore, the movement restriction mechanism including the locking mechanism and the actuation mechanism enables a restricted axial movement of the (at least one) inner member and the outer catheter relative to each other in the restricted moving state. The axial movement can be a simple displacement (i.e. a translatory movement) or a combined rotatory and translatory movement, preferably a regular or irregular oscillatory movement.

A preferred catheter system includes:
an outer catheter having an outer catheter shaft defining an outer catheter lumen capable of receiving (at least) one inner member,
(at least) one inner member being arranged within the outer catheter lumen of the outer catheter, the (at least one) inner member being relatively movable to the outer catheter in an unrestricted moving state, and
an axial movement restriction device including an actuation mechanism and a locking mechanism, wherein the axial movement restriction device is capable of restricting an axial movement of the (at least one) inner member in a restricted moving state compared to the axial movement in the unrestricted moving state.

The (at least one) inner member preferably is an inner tubular member or a wire-shaped inner member (or a combination thereof). The outer catheter lumen preferably is a tubular outer catheter lumen.

The combination of the locking mechanism and the actuation mechanism enables a variable usable length of the inner (tubular) member during operability.

The support catheter has a support catheter shaft defining a (tubular) lumen capable of receiving an inner (tubular) member. The support catheter has a proximal support catheter end and a distal support catheter end. The distal support catheter end is the end which is inserted into a human or animal body. The proximal support catheter end is the end, which is operated by an operator, in particular an interventional physician e.g. cardiologist or radiologist. The distal support catheter end may be straight-edged. A straight-edged distal support catheter end improves the pushability and avoids flaring.

The outer catheter, e.g. the support catheter, may be connected at its proximal end to an operational handle, preferably a handle enabling a pushing by an operator (namely a pushing handle). The handle may include visual, acoustic or haptic markings for improved length adjustability and handling by operators. The handle may have a gripping surface. The handle may be configured to simultaneously attach to the support catheter and the dilator.

The outer catheter, e.g. the support catheter, may further include a manifold member which can be connected to the operational handle, for example via a Luer connector. The manifold may have a shark fin shape.

The support catheter may further include one or more ports, e.g. flushing port(s), inflation and/or deflation port(s).

The catheter system may include as inner tubular member or in addition a dilator and/or a balloon catheter, e.g. a percutaneous transluminal angioplasty (PTA) catheter or a percutaneous transluminal coronary angioplasty (PTCA) catheter, and/or a catheter for applying a fluid medium, e.g. a contrast agent or a fluid including a drug, and/or a coronary catheter or a peripheral catheter (catheter for radiology interventions) and/or an otherwise interventional catheter.

The catheter system may include as wire-shaped inner member or in addition a needle or a guidewire.

The catheter system may be a multi-functional catheter system or interventional catheter system. Multi-Functional catheter system means that the support catheter can be accommodated simultaneously or consecutively with different inner members, like dilator and balloon catheter (e.g. PTA or PTCA catheter).

Preferably the catheter system includes:
a support catheter having a support catheter shaft defining a support catheter lumen capable of receiving a dilator and/or a balloon catheter, and
the dilator and/or the balloon catheter being arranged within the support catheter lumen of the support catheter, the dilator and/or the balloon catheter being relatively movable to the support catheter in an unrestricted moving state, and
an axial movement restriction device including an actuation mechanism and a locking mechanism, wherein the axial movement restriction device is capable of restricting an axial movement of the dilator and/or the balloon catheter in a restricted moving state compared to the axial movement in the unrestricted moving state.

The support catheter, the dilator and the balloon catheter (e.g. PTA or PTCA catheter) are each dimensionally adapted to each other.

The dilator has a dilator distal end, a dilator proximal end and a dilator shaft extending between the dilator distal end and the dilator proximal end. The dilator distal end includes a dilator tip, preferably made of a polymeric material. The tip can be a sharp or a blunt tip. The proximal dilator end may include a dilator manifold.

The balloon catheter has a balloon catheter distal end, a balloon catheter proximal end and a balloon catheter shaft extending between the balloon catheter distal end and the balloon catheter proximal end. The balloon catheter distal end includes a balloon (which is an inflatable member). The proximal balloon catheter end may include a balloon catheter manifold. The balloon may include radiopaque markers.

The catheter system may include a guidewire. A catheter system of this kind can be guided or pushed to the location of interest with the aid of the guide wire.

The axial movement restriction device including the actuation mechanism and the locking mechanism can be situated at or near the proximal outer catheter end, e.g. support catheter end. The axial movement restriction device including the actuation mechanism and the locking mechanism may be integrated in the handle. In such a case the handle is among its other functionalities used for restricting or unrestricting the inner member of the catheter system.

The axial movement restriction device including the actuation mechanism and the locking mechanism can be a separate mechanism which can be combined with any catheter system having an outer catheter and an inner member. Thus, the axial movement restriction device including an actuation mechanism and a locking mechanism is herein disclosed as well as an individual embodiment. The individual features of the locking mechanism and the actuation mechanism are described in detail elsewhere in the application.

In one embodiment the axial movement restriction device includes a locking mechanism including the locking mechanism and an actuation mechanism including the actuation mechanism, wherein the locking mechanism and the actuation mechanism are connected with each other or coupled to each other. The axial movement restriction device is capable of restricting an axial movement of at least one inner member, which can be inserted in the locking mechanism (mechanism). The axial movement restriction device or the actuation mechanism of the axial movement restriction device includes a connector (e.g. a Luer connector) for connecting the axial movement restriction device with an outer catheter (e.g. a support catheter) having a tubular outer catheter lumen capable of receiving an inner (tubular or wire-shaped) member. The axial movement restriction device includes a (tubular) receptor for the inner (tubular or wire-shaped) member, e.g. a catheter or a guidewire, or the locking mechanism and the actuation mechanism of the axial movement restriction device include a receptor for an inner (tubular or wire-shaped) member, e.g. a catheter or a guidewire.

The individual features of the locking mechanism (mechanism) and the actuation mechanism (mechanism) are described in detail elsewhere in the application.

Various embodiments of the locking mechanisms or locking mechanisms are possible. The locking mechanism can be situated in the locking mechanism, wherein the locking mechanism is connected with or coupled to the actuation mechanism.

The locking mechanism can include at least one clamping mechanism for locking the axial movement of the inner member in the locking state. In one embodiment the locking mechanism can include at least one clamping mechanism (suitable) for locking the axial movement of the inner member in the locking state.

In one embodiment the at least one clamping member can be a tapered clamp. Tapered clamps are pressed onto the inner member by a screw-in-cap that is connected via thread to the actuation mechanism (mechanism). The tapered clamps may be designed as two half shell clamps together fixing the inner member, preferably leaving open a channel for fluid flow when pressed onto the inner member.

In another embodiment the at least one clamping member can be a flexible clamping sleeve. The flexible clamping sleeve can be pressed onto the inner member by an outer cap. The sleeve flexibility might be created by material behavior and/or by slotted design.

In a further embodiment the at least one clamping member can have a clip-on design.

The clip-on design can consist of two parts together fixing the inner member.

In one embodiment the locking mechanism can be a hemostatic valve (also referred to as hemostasis valve). A hemostasis valve has a seal, which can be locked or unlocked, for example each time the inner member is introduced or extracted. The hemostatic valve may be connected to the actuation mechanism, for example via a Luer connection.

Various embodiments of the axial actuation mechanism are possible.

The actuation mechanism can be situated in an actuation mechanism, wherein the actuation mechanism is connected with or coupled to the locking mechanism.

In one embodiment the axial actuation mechanism being based on a spring-loaded actuation mechanism is preferably excluded.

In another embodiment the axial actuation mechanism can be based on a gas pressure spring.

In another embodiment the axial actuation mechanism can be based on a magnetic actuation mechanism, optionally combined with a damping mechanism.

In another embodiment the axial actuation mechanism is situated in a handle having a (lateral) lever. Via the lever an axial (forth and back) movement of the inner member can be achieved. The movement of the lever induced by the operators hand or finger(s), (e.g. a forth and back movement) can be transmitted into an axial movement of the inner member.

In another embodiment the axial actuation mechanism is situated in a handle having a (lateral) eyelet for the operators thumb or finger to (directly or indirectly) apply a movement, (e.g. a forth and back movement) of the operators thumb or finger to the inner member, e.g. using stroke limiting mechanisms.

The axial actuation mechanism can be a manual actuation mechanism. In a manual actuation mechanism, the axial actuation is manually applied by the operator supported by defined end stops to limit the stroke. The outer catheter is spatially kept in place by the operator's one hand while the inner member is axially moved back (towards the proximal outer catheter end) and forth (towards the distal outer catheter end) by the operator's other hand.

Alternatively, the axial actuation mechanism can be an electrically driven actuation mechanism, preferably being initiated by the operator.

The axial actuation mechanism can be a mechanism turning a rotation into axial translation or into a combined axial translation and rotation.

In one embodiment the axial actuation mechanism can include a threaded spindle. The threaded spindle turns rotation into axial translation with pitch and stroke according to requirements (axial movement limited by design of the thread) and back and forth with bidirectional rotation or unidirectional rotation. The axial movement can result from the rotation manually applied by the operator.

In another embodiment the axial actuation mechanism can be ball screw drive actuation mechanism. A ball screw drive actuation mechanism includes a bearing ball kept in place by an eyelet or slot in a housing of the actuation mechanism, combined with a thread-like groove where the bearing ball forces the outer catheter including the groove to axially travel in the intended manner.

The inner member might rotate during that actuation or not (depending on the catheter system requirements).

The combination of each aforementioned locking mechanism (mechanism) with each of the aforementioned axial actuation mechanism (mechanism) is herein disclosed.

Further described is an axial movement restriction device for restricting an axial movement of at least one inner member of a catheter, preferably the inner member being a balloon or a tubular or wire-shaped inner member, like a guidewire or dilator.

The axial movement restriction device for restricting an axial movement of at least one inner member being arranged in (a catheter lumen of) a catheter, wherein the axial movement restriction device includes a locking mechanism and an actuation mechanism. The locking mechanism and the actuation mechanism are connected with each other or coupled to each other.

The axial movement restriction device can include a locking mechanism including the locking mechanism and an actuation mechanism including the actuation mechanism, wherein the locking mechanism and the actuation mechanism are connected with each other or coupled to each other. For example, the actuation mechanism can include an axial actuation mechanism which converts a rotary motion into a translatory motion of the locking mechanism. Thus, the actuation mechanism can include a rotation-translation gear for converting a rotational movement into a translatory movement.

The axial movement restriction device further includes a receptor for the inner member (e.g. a balloon or a tubular or wire-shaped inner member, like a guidewire or dilator). Preferably, the locking mechanism and the actuation mechanism of the axial movement restriction device include a receptor for the inner member (e.g. a balloon or a tubular or wire-shaped inner member, like a guidewire or dilator).

The axial movement restriction device is capable of restricting an axial movement of at least one inner member, which can be inserted in the axial movement restriction device and/or the locking mechanism.

The axial movement restriction device and/or the actuation mechanism of the axial movement restriction device can include a connector for connecting the axial movement restriction device and/or the actuation mechanism with a (outer) catheter having a (tubular outer) catheter lumen capable of receiving an inner member (e.g. a balloon or a tubular or wire-shaped inner member, like a guidewire or dilator). The connector may be a Luer connector.

A preferred method for operating a catheter system includes:
an outer catheter having an outer catheter shaft defining an outer catheter lumen capable of receiving (at least) one inner member, and
(at least) one inner member arranged within the outer catheter lumen of the outer catheter,
restricting the axial movement of the at least one inner member via an axial movement restriction device including an actuation mechanism and a locking mechanism in a restricted moving state compared to an unrestricted moving state.

By restricting the axial movement of the (at least one) inner member a better performance can be achieved.

The method can further include a step of unrestricting the axial movement of the (at least one) inner member via the axial movement restriction device in the unrestricted moving state and optionally moving the inner member within the outer catheter lumen of the outer catheter.

Also described is a catheter system as described above or a method for operating a catheter system wherein:
the (at least one) inner member can be an inner tubular member or an inner wire-shaped member;
the outer catheter can be a support catheter and/or the (at least one) inner member can be a dilator, a balloon catheter, a catheter for applying a drug or a fluid medium a coronary catheter, a peripheral catheter and/or any other interventional catheter;
the inner member can be a percutaneous transluminal angioplasty catheter or a percutaneous transluminal coronary angioplasty catheter;
the locking mechanism can be a clamping mechanism for locking the (at least one) inner member including at least one clamp. The at least one clamp can be made up by several parts, preferably by two half shell clamps, arranged together as one clamp;
the actuation mechanism is a manual actuation mechanism or an electrically driven actuation mechanism including a drive unit;
the actuation mechanism can be spindle driven actuation mechanism or a bayonet mount driven actuation mechanism. Alternatively, the actuation mechanism includes a groove and a bearing ball being capable of sliding within the groove;
the actuation mechanism is not based on a spring-loaded actuation mechanism;
the axial movement is a translatory movement or a combined rotatory and translatory movement;
the actuation mechanism includes a means for generating vibrations (e.g. having a frequency of 1 Hz to 100 kHz, preferably 5 Hz to 15 kHz, more preferably 5 Hz to 500 Hz) and a transmission means for transmitting said vibrations to the inner member;
the (at least one) inner tubular member (axially) oscillates, preferably with frequency of 1 Hz to 100 kHz, more preferably 5 Hz to 15 kHz, most preferably 5 Hz to 500 Hz.

By using the actuation mechanism including a means for generating vibrations having a frequency of 5 Hz or more (and less than 100 kHz, 15 kHz or 500 Hz) mechanical manipulations clinically desired can be facilitated when addressing a stenosis or chronic total occlusion compared the inner member being moved manually by an operator.

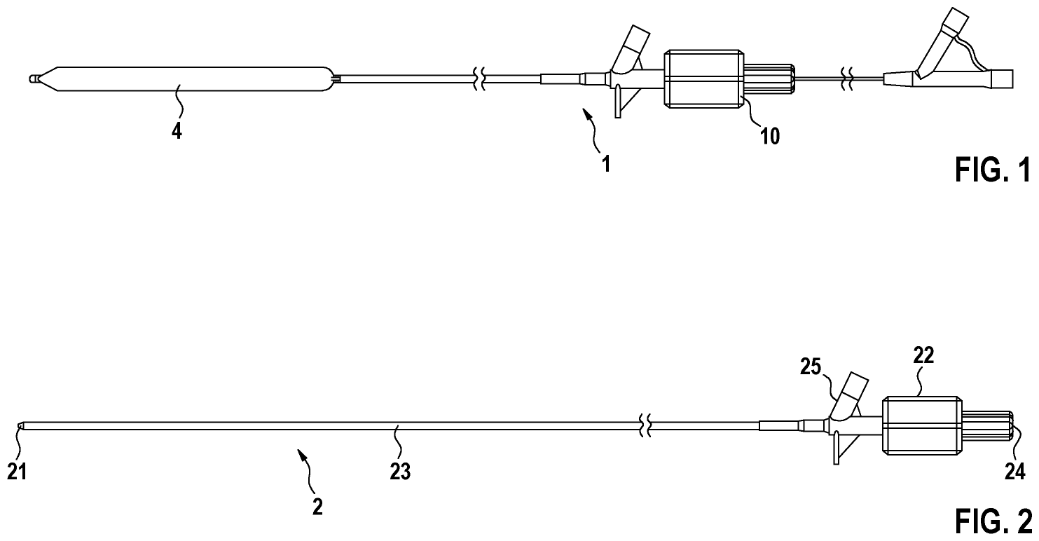

FIG. 1 shows a catheter system 1 including an outer catheter (e.g. a support catheter). The outer catheter shaft defines a tubular outer catheter lumen capable of receiving one inner tubular member. The catheter system 1 includes an axial movement restriction device 10. Here the inner tubular member shown is a balloon catheter 4 (e.g. PTA catheter), which is arranged within the tubular outer catheter lumen of the outer catheter. However instead of the balloon catheter a dilator can be used instead. The inner tubular member is relatively movable to the outer catheter in an unrestricted moving state.

In FIG. 2 a support catheter 2 is shown. The support catheter 2 has a support catheter distal end 21, a support catheter proximal end 24 and a support catheter shaft 23 extending between the support catheter distal end 21 and the support catheter proximal end 24. The proximal support catheter end 24 may include a support catheter handle 22 being connected to a support catheter manifold 25. The manifold can include a port.

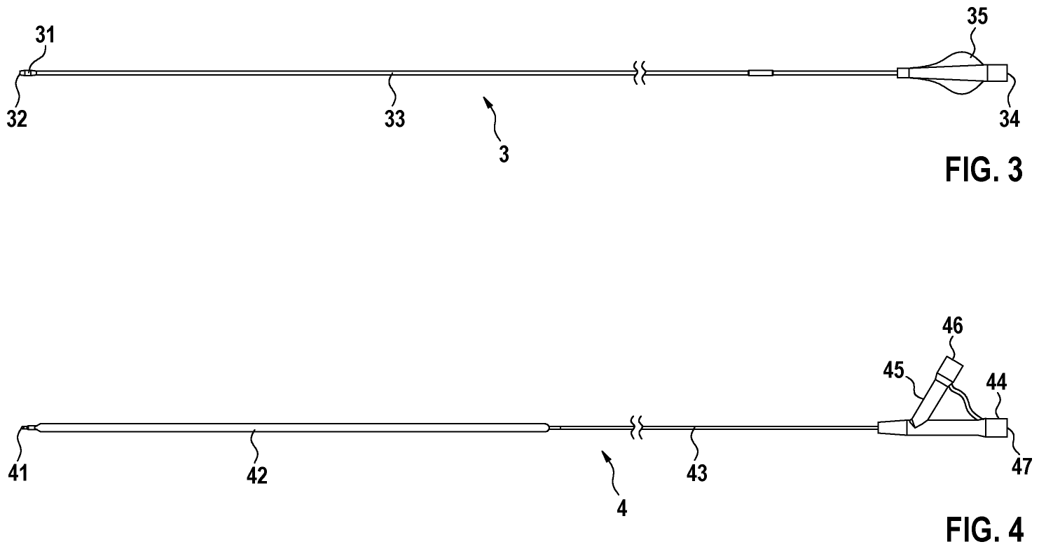

FIG. 3 shows a dilator 3 having a dilator distal end 31, a dilator proximal end 34 and a dilator shaft 33 extending between the dilator distal end 31 and the dilator proximal end 34. The dilator distal end 31 includes a dilator tip 32. The tip can be a sharp or a blunt tip. The proximal dilator end 34 may connected with a dilator manifold 25.

FIG. 4 shows a balloon catheter 4 having a balloon catheter distal end 41, a balloon catheter proximal 44 end and a balloon catheter shaft 43 extending between the balloon catheter distal end 41 and the balloon catheter proximal end 44. The balloon catheter distal end 41 includes a balloon 42 (which is an inflatable member). The proximal balloon catheter end 44 may include a balloon catheter manifold 45. The manifold may include an inflation/deflation port 46 and a guidewire port 47.

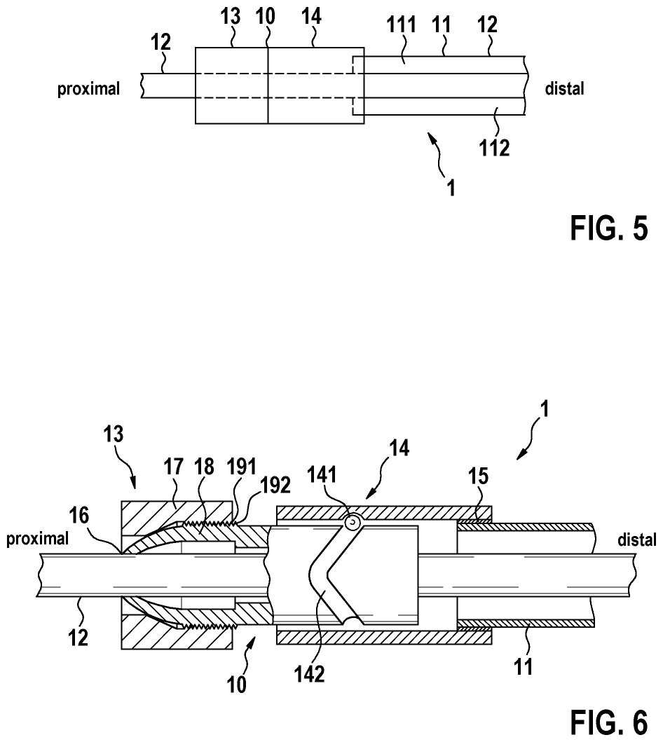

FIG. 5 shows a catheter system 1 including an outer catheter 11 having an outer catheter shaft 112 defining an outer catheter lumen 111 capable of receiving at least one inner member 12, and at least one inner member 12 being arranged within the outer catheter lumen 111 of the outer catheter 11. The catheter system 1 further includes an axial movement restriction device 10 including an actuation mechanism 14 and a locking mechanism 13. The axial movement restriction device 10 is capable of restricting an axial movement of the at least one inner member 12 in a restricted moving state compared to the axial movement in the unrestricted moving state. The actuation mechanism 14 is connected to the locking mechanism 13.

FIG. 6 shows another embodiment of a catheter system 1 including an axial movement restriction device 10 including an actuation mechanism 14 and a locking mechanism 13 of the catheter system in a cross-sectional side view. The actuation mechanism includes a bearing ball 141 kept in place by a bearing ball receptacle (e.g. an eyelet or slot) in a housing surrounding the actuation mechanism combined with a (thread-like) groove 142 where the bearing ball 141 forces the part with the groove 142 to axially travel in the intended manner. The axial movement results from the rotation manually applied by the clinical user. The groove may be an oscillating groove. The outer catheter 11 (only partly shown) is connected to the actuation mechanism 14 of the axial movement restriction device 10 via a connection mechanism 15, e.g. a Luer connector. Preferably, the bearing ball receptacle is connected to the outer catheter 11. The outer catheter 11 (only partly shown) is spatially kept in place (users first hand) while the inner member 12 is axially propagating driven by the handle rotated by users second hand. The inner catheter might rotate during that actuation or not according to product requirements and implemented embodiment. The actuation mechanism 14 is connected to the locking mechanism 13. The locking mechanism includes a stiff member 17 having a first thread part 191 and a sleeve 18 having a second thread part 192. The sleeve has 18 a soft tip 16, e.g. a tip made of an elastomer.

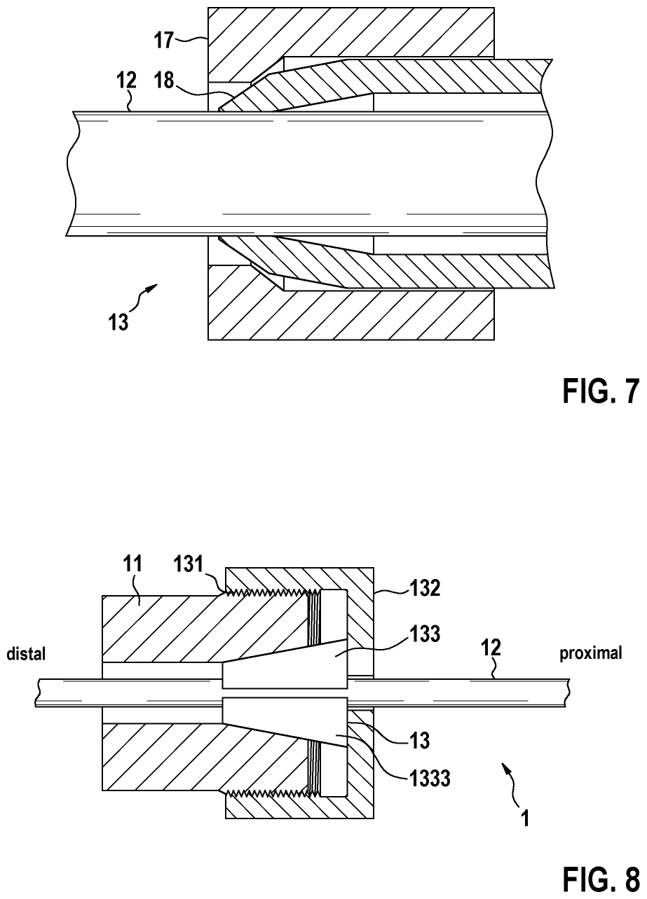

FIG. 7 shows an embodiment of a locking mechanism 13 in a longitudinal cross-sectional side view. The at least one clamping member can include a flexible clamping sleeve 18. The flexible sleeve is pressed onto the inner member 12 by an outer cap 17.

FIG. 8 shows an embodiment of a catheter system 1 with a locking mechanism 13 in a cross-sectional side view. The at least one clamping member 133 can be a tapered clamp. The clamping member 133 may be made of an elastomer. The tapered clamp is pressed onto the inner member 12 by a screw-in-cap 132 that is connected via thread 131 to the outer catheter shaft 11. The tapered clamp may be designed as two half shell clamps 1333. The two half shell clamps 1333, arranged together as one clamp, can be pressed onto the inner member 12 by the screw-in-cap 132. The two half shell clamps leave an open channel for fluid flow when pressed onto the inner member 12. The half shell clamps may be directly connected to the screw-in-cap 132 or are formed integrally with the screw-in-cap 132.

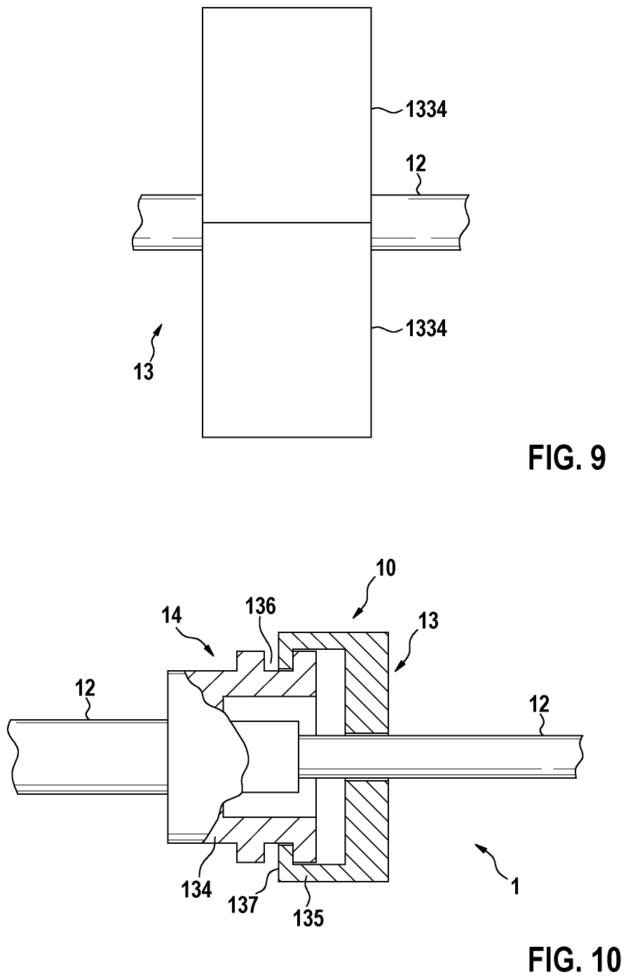

FIG. 9 shows a further embodiment of a locking mechanism 13 in a side view which is a clip-on mechanism with two clip-on half shells 1334 pressed onto the inner member 12. The releasable clip-on mechanism based on e.g. frictional forces or a snap hook design. The clip-on half shells may include an elastomer on the side of the half shells facing the inner member 12.

FIG. 10 shows another embodiment of a catheter system 1 with an axial movement restriction device 10 including an actuation mechanism 14 and a locking mechanism 13 in a side view. The axial actuation mechanism 14 and the locking mechanism 13 are connected to each other and work together. The axial actuation mechanism 14 is manually applied by the clinical user supported by defined end stops to limit the stroke. The outer catheter 11 is spatially kept in place (by user's first hand) while the inner member 12 is axially propagating back and forth by a user's second hand. The locking mechanism 13 includes of a two-part stroke including a first part of an axial two way stroke limiter 134 including a depression 136 and a second part of an axial two way stroke limiter 135 (as counterpart) including a protrusion 137 arranged together with the groove 136 of the first part of an axial two way stroke limiter 134. The second part of an axial two-way stroke limiter 135 has a circumferential inner ledge engaging with the first part of an axial two-way stroke limiter 134 e.g. via a groove to limit axial stroke in two directions. The first part of the axial two-way stroke limiter 134 is connected to or integral part of the outer catheter 11. The second part of the axial two-way stroke limiter 135 is connected to the inner member 12. The locking mechanism 13 is formed integrally with the second part of an axial two-way stroke limiter 135. The locking mechanism is releasably being clamped or snapped on the inner catheter 12 preferably during clinical procedure.

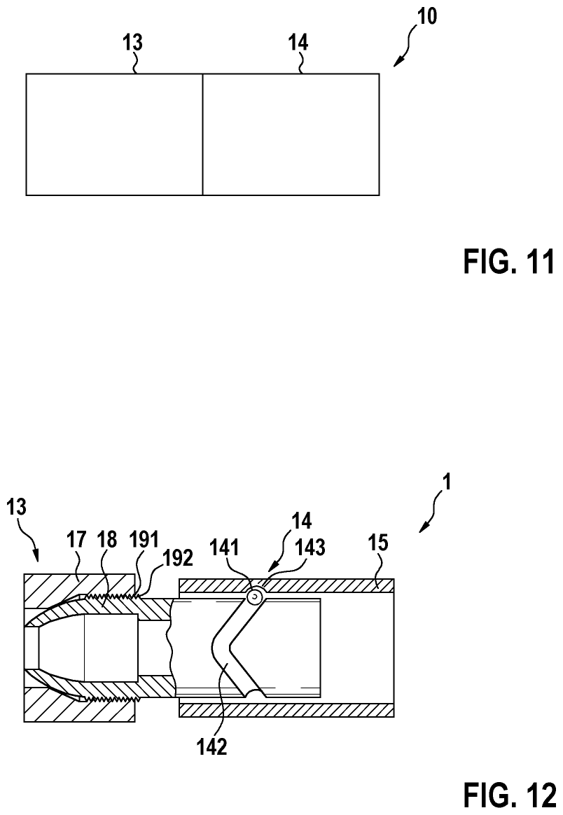

FIG. 11 shows an embodiment of an axial movement restriction device 10 including an actuation mechanism 14 and a locking mechanism 13. The actuation mechanism 14 is connected to the locking mechanism 13. The axial movement restriction device 10 can include a locking mechanism including the locking mechanism 13 and an actuation mechanism including the actuation mechanism 14, wherein the locking mechanism and the actuation mechanism are connected with each other or coupled to each other. The axial movement restriction device 10 is capable of restricting an axial movement of at least one inner member, which can be inserted in the locking mechanism (mechanism). The axial movement restriction device 10 or the actuation mechanism of the axial movement restriction device 10 may include a connector for connecting the axial movement restriction device 10 with an outer catheter (e.g. a support catheter) having an outer catheter lumen capable of receiving an inner (tubular or wire-shaped) member. The axial movement restriction device 10 includes a receptor for the inner (tubular or wire-shaped) member, e.g. a catheter or a guidewire. Preferably the locking mechanism and the actuation mechanism of the axial movement restriction device 10 include a receptor for an inner (tubular or wire-shaped) member, e.g. a catheter or a guidewire.

FIG. 12 shows another embodiment of an axial movement restriction device including an actuation mechanism 14 and a locking mechanism 13 in a cross-sectional side view. The actuation mechanism 14 includes a bearing ball 141 kept in place by bearing ball receptacle 143 (e.g. eyelet or slot) situated in an actuation mechanism housing which is combined with a thread-like groove 142 where the bearing ball 141 forces the part with the groove 142 to axially travel in the intended manner. The axial movement results from the rotation applied by an operator. The groove may be an oscillating groove. The actuation mechanism 14 of the axial movement restriction device 10 can be connectable to an outer catheter (not shown) via a connection mechanism 15, e.g. a Luer connector. Preferably, the bearing ball receptacle 143 is connectable to the outer catheter. The actuation mechanism 14 is connected to the locking mechanism 13. The locking mechanism includes a stiff member 17 having a first thread part 191 and a sleeve 18 having a second thread part 192. The sleeve has a soft tip 16, e.g. a tip made of an elastomer.

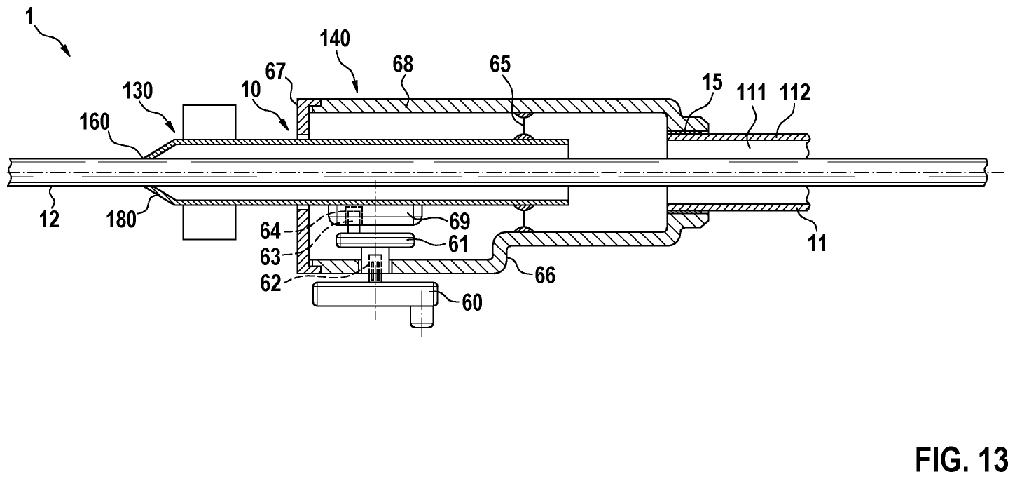

FIG. 13 shows another embodiment of a catheter system 1 including an axial movement restriction device 10 in a cross-sectional side view. The axial movement restriction device includes a locking mechanism 130 including a locking mechanism and an actuation mechanism 140 including an actuation mechanism, wherein the locking mechanism 130 and the actuation mechanism 140 are connected with each other or coupled to each other and interact with each other. Thus, the actuation mechanism is coupled to the locking mechanism. The actuation mechanism 140 includes an axial actuation mechanism. In the axial actuation mechanism shown in FIG. 13 a rotary motion is converted into a translatory motion of the locking mechanism 130. The actuation mechanism 140 may include a rotation-translation gear for converting a rotational movement into a translatory movement. However, the actuation mechanism 140 could also include an axial actuation mechanism, wherein a translational movement is converted directly or indirectly into a translatory movement.

The catheter system 1 includes an outer catheter 11 (e.g. a guiding catheter) having an outer catheter shaft 112 which defines a tubular outer catheter lumen 111 capable of receiving a (tubular) inner member 12 (e.g. a dilator). The axial movement restriction device 10, specifically the actuation mechanism 140, is connected via a connection mechanism 15 (e.g. a Luer connector) with the outer catheter 11.

In detail, the locking mechanism 130 may include a sleeve 180. The sleeve 180 may include a flexible tip 160 at its proximal sleeve part. The sleeve 180 may include a seal 65 at its distal sleeve part. The seal 65 enables an axial lifting movement. The seal 65, e.g. a sealing ring, a sealing membrane or sealing lip, may have a sealing function for a fluid and at the same time acts as a guiding mechanism. The sleeve 180 may further include a notch 64 in its middle sleeve part, being situated between the proximal sleeve part and the distal sleeve part or the notch may be situated at a notch mechanism 69 (e.g. disk) being directly attached to the middle sleeve part. The notch 64 may have a spiral track for adjusting the axial lifting movement. The locking mechanism 130 and the actuation mechanism 140 are coupled to each other via the seal 65.

The actuation mechanism 140 may include a first rotating mechanism 60, a drive shaft 62 (with or without gear transmission), a second rotating mechanism 61 and a housing 66. The housing 66 may be a two-part housing including a main housing body 68 and a housing cap 67. The main housing body 68 and a housing cap 67 can be assembled, e.g. clipped together. The housing cap 67 can act as guiding support for the sleeve 18.

The first rotating mechanism 60 is preferably a rotary wheel or a crank (being operable with one finger or thumb). The second rotating mechanism 61 is preferably smaller than the first rotating mechanism 60. The second rotating mechanism 61 may include a pin 63. The first rotating mechanism 60 can be located (partially) inside or outside the housing 66.

The movement of the first rotating mechanism 60 is transferred via the drive shaft 62 with or without gear transmission to the second rotating mechanism 61. The pin 63 of the second rotating mechanism 61 interacts with the notch 64 and thus causes a translatory movement of the sleeve 18. Thus, the axial actuation of this axial movement restriction device 10 is triggered by converting a rotary motion of the first rotating mechanism 60, e.g. a rotary wheel or crank, of the actuation mechanism 140 into a translatory motion of the sleeve 18. The flexible tip of the sleeve 18 is pressed onto the inner member 12 and thus the inner member is locked.

Instead of the first rotating mechanism 60, the drive shaft 62 and the second rotating mechanism 61 with the pin 63 interacting with the notch 64 the actuation mechanism 140 can include any actuation mechanism (excepting spring based mechanisms) converting a rotary or translatory movement into a translatory motion of the sleeve 18. This can be for example a mechanism where an operator needs to press a button or operate a lever.

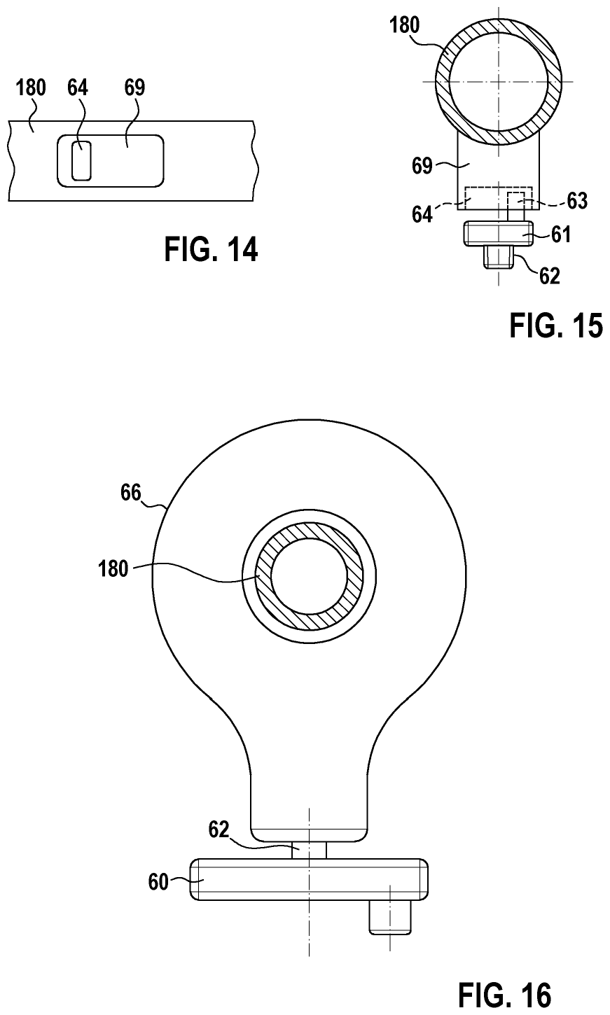

FIG. 14 shows a part of the axial movement restriction device 10 of FIG. 13 in a cross-sectional view. This part shows the sleeve 180 being connected or coupled to the notch mechanism 69 (e.g. disk) including the notch 64.

FIG. 15 shows a part of the axial movement restriction device 10 of FIG. 13 in a cross-sectional front view. This part shows the sleeve 180 being connected or coupled to the notch mechanism 69 (e.g. disk) including the notch 64. The notch 64 interacts with the pin 63, which is mounted on the second rotating mechanism 61.

FIG. 16 shows a part of the axial movement restriction device 10 of FIG. 13 in a cross-sectional front view. This part shows the sleeve 180 being mounted in the housing 66. Here a part of the drive shaft 62 and the first rotating mechanism 60 is located outside the housing. Alternatively, the drive shaft 62 can be located within the housing 66. Alternatively, a part of or the whole first rotation mechanism 60 can be located within the housing 66, then an opening for a finger or thumb of the operator is necessary within the housing 66 through which the first rotation mechanism 60 can be controlled.

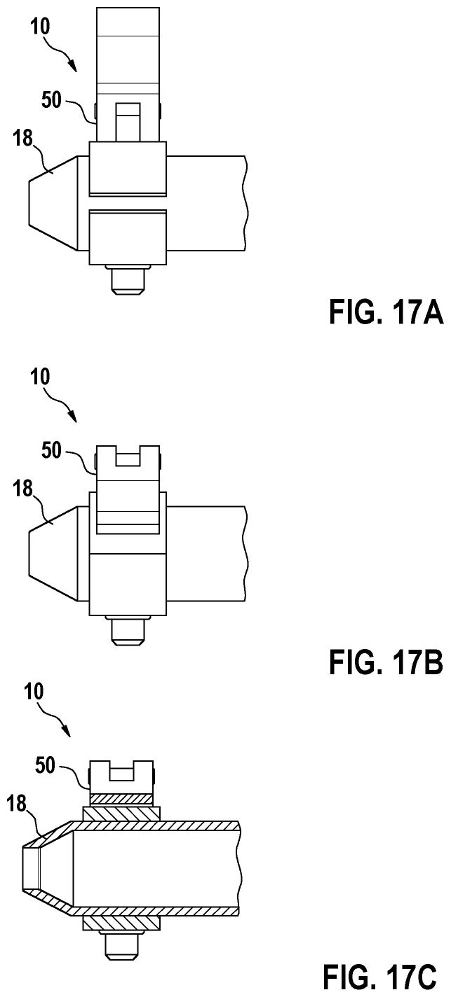

FIGS. 17A, 17B and 17C show another embodiment of an axial movement restriction device 10 including a force application mechanism 50 which is configured to apply a force to the sleeve 18 in circumferential direction. By applying a circumferential force to the sleeve 18 the axial movement restriction device 10 is capable of restricting an axial movement of the at least one inner member in a restricted moving state compared to the axial movement in an unrestricted moving state. The force application mechanism 50 may be a ring-shaped or tubular-shaped force application mechanism 50. The force application mechanism 50 may be a quick release clamp e.g. including a ring-shaped or tubular-shaped collar. The type of mechanism used by the quick-release clamp can include a lever, a trigger, or a spring. A quick-release clamp is configured to be used one-handed. FIG. 17A shows a side view of the axial movement restriction device 10, wherein the force application mechanism 50 is in an unrestricted moving state, for example a lever which is fastened by pushing it towards the ring-shaped collar of the quick-release clamp. FIG. 17B shows a side view of the axial movement restriction device 10, wherein the force application mechanism 50 is in a restricted moving state, for example a lever which is loosened by pulling the lever away from the ring-shaped collar of the quick-release clamp. FIG. 17 shows a cross sectional side view of the axial movement restriction device 10 including a sleeve 18 and a force application mechanism 50.

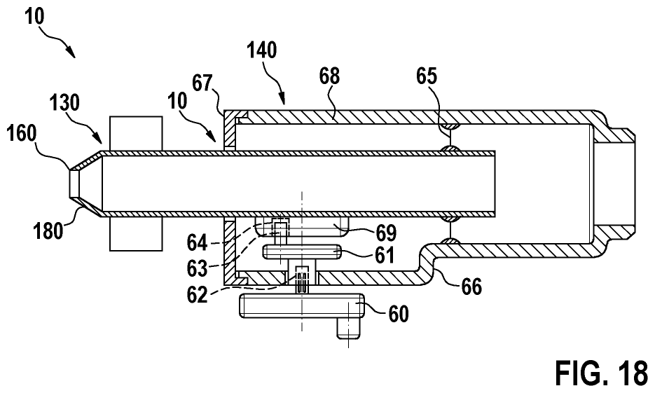

FIG. 18 shows the axial movement restriction device 10 as described in FIG. 13 in a cross-sectional side view. The axial movement restriction device 10 includes a locking mechanism 130 including a locking mechanism and an actuation mechanism 140 including an actuation mechanism, wherein the locking mechanism 130 and the actuation mechanism 140 are connected with each other or coupled to each other. Thus, the actuation mechanism is coupled to the locking mechanism. The actuation mechanism 140 includes an axial actuation mechanism which converts a rotary motion into a translatory motion of the locking mechanism 130. Preferably, the actuation mechanism 140 includes a rotation-translation gear.

### LIST OF REFERENCE NUMBERS

1 catheter system
10 axial movement restriction device
11 outer catheter
111 outer catheter lumen
112 outer catheter shaft
12 inner member
13 locking mechanism
130 locking mechanism
131 thread
132 screw in cap
133 clamping member
1333 half shell clamp
1334 clip-on half shells
134 first part of axial two-way stroke limiter
135 second part of two-way stroke limiter
136 depression
137 protrusion
14 Actuation mechanism
140 Actuation mechanism
141 bearing ball
143 bearing ball receptacle
142 groove
15, 150 connection mechanism
16, 160 tip
17 stiff member
171 cap
18, 180 sleeve
191 first thread part
192 second thread part
2 support catheter
21 support catheter distal end
22 support catheter handle
23 support catheter shaft
24 support catheter proximal end
25 support catheter manifold
3 dilator
31 dilator distal end
32 dilator tip
33 dilator shaft
34 catheter proximal end
35 dilator manifold
4 balloon catheter
41 balloon catheter distal end
42 balloon
43 balloon catheter shaft
44 balloon catheter proximal end
45 balloon catheter manifold
46 inflation/deflation port
47 guidewire port
50 force application mechanism
60 first rotating mechanism
61 second rotating mechanism
62 drive shaft
63 pin
64 notch
65 seal
66 housing
67 housing cap
68 main housing body
69 notch mechanism

1. A catheter system comprising or consisting of:
an outer catheter comprising an outer catheter shaft defining an outer catheter lumen;
at least one inner member being arranged within the lumen, the at least one inner member being relatively movable with respect to the outer catheter in an unrestricted moving state, and
an axial movement restriction device comprising an actuation mechanism and a locking mechanism, wherein the axial movement restriction device is configured to restrict axial movement of the at least one inner member in a restricted moving state and permit axial movement in the unrestricted moving state, and wherein the actuation mechanism comprises a rotation-translation gear, a ball screw drive actuation mechanism or a spindle driven actuation mechanism.

2. The catheter system of claim 1, wherein the actuation mechanism and the locking mechanism are connected or coupled to each other.

3. The catheter system of claim 1, wherein the axial movement restriction device comprises a connector that connects the axial movement restriction device with the outer catheter.

4. The catheter system of claim 1, wherein the axial movement restriction device comprises a receptor for the inner member.

5. The catheter system of claim 1, wherein the outer catheter is connected at its proximal end to a handle.

6. The catheter system of claim 5, wherein the axial movement restriction device is integrated in the handle.

7. The catheter system of claim 1, wherein the outer catheter is a support catheter.

8. The catheter system of claim 1, wherein the at least one inner member is an inner tubular member or an inner wire-shaped member.

9. The catheter system of claim 1, wherein the at least one inner member is a dilator and/or a balloon catheter.

10. (canceled)

11. The catheter system of claim 1, wherein the locking mechanism comprises at least one clamp.

12. The catheter system of claim 11, wherein the at least one clamp comprises two half shell clamps arranged together as one clamp.

13. The catheter system of claim 1, wherein the actuation mechanism is a manual actuation mechanism.

14. The catheter system of claim 1, wherein the actuation mechanism is an electrically driven actuation mechanism comprising a drive unit.

15. The catheter system of claim 1, wherein the actuation mechanism comprises a ball screw drive actuation mechanism, which comprises an oscillating thread.

16. An axial movement restriction device for restricting an axial movement of at least one inner member arranged in an outer catheter lumen of an outer catheter, catheter, comprising:
a locking mechanism and an actuation mechanism connected or coupled with each other;
a connector configured to connect the axial movement restriction device with an outer catheter having a tubular outer catheter lumen capable of receiving an inner member; and
a tubular receptor for the inner member; wherein the actuation mechanism comprises
a rotation-translation gear, a ball screw drive, or a spindle driven actuation mechanism.

17. (canceled)

18. (canceled)

19. (canceled)

20. The catheter system of claim 1, wherein the at least one inner member being is relatively movable with respect to the outer catheter via a translatory movement or a combined rotatory and translatory movement.

21. The catheter system of claim 14, wherein the drive unit is configured to oscillate the at least one inner tubular member with a frequency of 1 Hz to 100 KHz.

22. The catheter system of claim 21, wherein the drive unit is configured to oscillate the at least one inner tubular with a frequency of 5 Hz to 15 kHz.

23. The catheter system of claim 21, wherein the drive unit is configured to oscillate the at least one inner tubular with a frequency of 5 Hz to 500 Hz.

24. The catheter system of claim 1, wherein the catheter comprises a percutaneous transluminal angioplasty catheter, a catheter configured to apply a drug or a fluid medium, a radiology intervention catheter, or a cardiology intervention catheter.
